(19) United States
(12) Patent Application Publication
(10) Pub. No.: US 2026/0038113 A1
(43) Pub. Date: Feb. 5, 2026

(54) METHOD AND APPARATUS FOR DETERMINING ENDOBRONCHIAL TUBERCULOSIS TYPING, AND DEVICE

(71) Applicants: Shanghai University of Engineering Science, Shanghai (CN); Jiangxi Chest Hospital, Nanchang City (CN)

(72) Inventors: Lingyan HU, Shanghai (CN); Yuqiao XIN, Southampton (GB); Zhongshu CHEN, Nanchang City (CN); Xueyu ZHANG, Nanchang City (CN); Hengkai RUAN, Nanchang City (CN); Jian LIN, Nanchang City (CN); Bin WANG, Nanchang City (CN); Dongmei XU, Shanghai (CN)

(21) Appl. No.: 19/021,786

(22) Filed: Jan. 15, 2025

(30) Foreign Application Priority Data
Aug. 2, 2024 (CN) 202411058052.4

(51) Int. Cl. G06T 7/00; A61B 1/00; A61B 1/267; G16H 50/20

(52) U.S. Cl. CPC G06T 7/0012; A61B 1/000096; A61B 1/2676; G16H 50/20; G06T 2207/10068; G06T 2207/20081; G06T 2207/20084; G06T 2207/30004

(57) ABSTRACT

Provided are a method and an apparatus for determining endobronchial tuberculosis typing, and a device, and relates to the field of artificial intelligence-assisted diagnosis. The method includes: obtaining a dataset, where the dataset includes an endobronchial endoscopic image sample; constructing an endobronchial tuberculosis diagnostic model, where the endobronchial tuberculosis diagnostic model is an endobronchial tuberculosis diagnostic model that is based on a ResNet34 framework and that incorporates multi-head self-attention and depthwise separable convolution; training the endobronchial tuberculosis diagnostic model based on the dataset; and inputting a bronchoscopy image of a user into a trained endobronchial tuberculosis diagnostic model to obtain the endobronchial tuberculosis typing. According to this application, intelligent diagnosis of endobronchial tuberculosis can be implemented through an artificial intelligence-assisted diagnostic system, so that misdiagnosis and missed diagnosis of endobronchial tuberculosis can be effectively reduced.

### CROSS REFERENCE TO RELATED APPLICATION

This patent application claims the benefit and priority of Chinese Patent Application No. 2024110580524, filed with the China National Intellectual Property Administration on Aug. 2, 2024, the disclosure of which is incorporated by reference herein in its entirety as part of the present application.

### TECHNICAL FIELD

This application relates to the field of artificial intelligence-assisted diagnosis, and in particular, relates to a method and apparatus for determining endobronchial tuberculosis typing, and a device.

### BACKGROUND

Tuberculosis is one of the top infectious disease killers worldwide. Every year, tens of millions of people are affected with tuberculosis. Every day, more than 3,500 people are died from this preventable and curable disease. According to the Global Tuberculosis Report 2023 released by the World Health Organization (WHO) on Nov. 7, 2023, China has the third highest number (748,000 cases, where 95% CI: 634,000 to 872,000) of new tuberculosis cases in 2022. Among these cases, about 250,000 are diagnosed as patients with endobronchial tuberculosis every year. In China, more than 60% of cases have serious complications, including pulmonary atelectasis, endobronchial stenosis, and lung function impairment, as well as lung destruction. In some cases, these complications necessitate lobectomy for treatment. As endobronchial tuberculosis lacks specific clinical symptoms and imaging manifestations, an experienced doctor is required to provide an accurate diagnosis for endobronchial endoscopy. In this case, endobronchial tuberculosis is frequently misdiagnosed and underdiagnosed, and consequently the optimal opportunity for treatment is missed. Therefore, if endobronchial tuberculosis is intelligently diagnosed via an artificial intelligence-assisted diagnostic system, misdiagnosis and missed diagnosis of endobronchial tuberculosis can be effectively reduced, facilitating early detection and treatment, and mitigating a risk of tuberculosis transmission.

### SUMMARY

An objective of this application is to provide a method and an apparatus for determining endobronchial tuberculosis typing, and a device, to effectively reduce misdiagnosis and missed diagnosis of endobronchial tuberculosis by intelligently diagnosing endobronchial tuberculosis via an artificial intelligence-assisted diagnostic system.

To achieve the above objective, this application provides the following technical solutions.

According to a first aspect, this application provides a method for determining endobronchial tuberculosis typing, including:
obtaining a dataset, where the dataset includes an endobronchial endoscopic image sample;
constructing an endobronchial tuberculosis diagnostic model, where the endobronchial tuberculosis diagnostic model is an endobronchial tuberculosis diagnostic model that is based on a ResNet34 framework and that incorporates multi-head self-attention and depthwise separable convolution;
training the endobronchial tuberculosis diagnostic model based on the dataset; and
inputting a bronchoscopy image of a user into a trained endobronchial tuberculosis diagnostic model to obtain the endobronchial tuberculosis typing.

Optionally, the training the endobronchial tuberculosis diagnostic model based on the dataset specifically includes the following steps:
inputting the endobronchial endoscopic image sample into the endobronchial tuberculosis diagnostic model to obtain a model output;
calculating a difference between the model output and a true label by using a cross-entropy loss function to obtain a loss;
calculating a gradient of the loss with respect to a parameter of the endobronchial tuberculosis diagnostic model, and propagating the gradient of the loss from an output layer to an input layer through a chain rule; and
updating, by an optimizer, the parameter of the endobronchial tuberculosis diagnostic model based on the gradient of the loss, to obtain the trained endobronchial tuberculosis diagnostic model.

Optionally, the endobronchial tuberculosis diagnostic model specifically includes:
a 7×7 convolutional layer, a pooling layer, a first residual module group, a second residual module group, a third residual module group, a fourth residual module group, a global average pooling layer, and a fully connected layer, where
the 7×7 convolutional layer, the pooling layer, the first residual module group, the second residual module group, the third residual module group, the fourth residual module group, the global average pooling layer, and the fully connected layer are sequentially connected.

Optionally, the first residual module group includes a first ordinary residual block, a second ordinary residual block, and a third ordinary residual block, where each of the first ordinary residual block, the second ordinary residual block, and the third ordinary residual block includes two 3×3 convolutional layers, and the first ordinary residual block, the second ordinary residual block, and the third ordinary residual block are sequentially connected.

The second residual module group includes a first depthwise separable convolution residual block, a fourth ordinary residual block, a fifth ordinary residual block, and a sixth ordinary residual block, where each of the fourth ordinary residual block, the fifth ordinary residual block, and the sixth ordinary residual block includes two 3×3 convolutional layers.

The first depthwise separable convolution residual block, the fourth ordinary residual block, the fifth ordinary residual block, and the sixth ordinary residual block are sequentially connected.

The third residual module group includes a second depthwise separable convolution residual block, a seventh ordinary residual block, an eighth ordinary residual block, a ninth ordinary residual block, a tenth ordinary residual block, and an eleventh ordinary residual block, where each of the seventh ordinary residual block, the eighth ordinary residual block, the ninth ordinary residual block, the tenth ordinary residual block, and the eleventh ordinary residual block includes two 3×3 convolutional layers.

The second depthwise separable convolution residual block, the seventh ordinary residual block, the eighth ordinary residual block, the ninth ordinary residual block, the tenth ordinary residual block, and the eleventh ordinary residual block are sequentially connected.

The fourth residual module group includes a third depthwise separable convolution residual block, a first multi-head self-attention mechanism residual block, and a second multi-head self-attention mechanism residual block.

The third depthwise separable convolution residual block, the first multi-head self-attention mechanism residual block, and the second multi-head self-attention mechanism residual block are sequentially connected.

Optionally, the first ordinary residual block, the second ordinary residual block, the third ordinary residual block, the fourth ordinary residual block, the fifth ordinary residual block, the sixth ordinary residual block, the seventh ordinary residual block, the eighth ordinary residual block, the ninth ordinary residual block, the tenth ordinary residual block, and the eleventh ordinary residual block are all calculated according to the following formula:

$$y = F ( x , \{ {W}_{i} \} ) + {W}_{1 \times 1} * x ,$$

where

y represents an output, F(x,{Wᵢ})=ReLU(BN(W₂*ReLU(BN(W₁*x)))), x represents an input, BN represents a batch normalization operation, ReLU represents nonlinear calculation, i=1 and 2 in Wᵢ, W₁ represents a first convolution operation and a weight, W₂ represents a second convolution operation and a weight, and W_{1×1} represents a 1×1 convolution operation and a weight.

Optionally, the first depthwise separable convolution residual block, the second depthwise separable convolution residual block, and the third depthwise separable convolution residual block are all calculated according to the following formula:

where

$$y = F ( x , \{ {W}_{i} \} ) + {W}_{p w} * ( {W}_{d w} * x ) ,$$

W_{pw} represents a pointwise convolution operation, and W_{dw} represents a depthwise convolution operation.

Optionally, both the first multi-head self-attention mechanism residual block, and the second multi-head self-attention mechanism residual block are calculated according to the following formula:

$$y = ReLU ( BN ( {W}_{3 \times 3} * M H S A ( X ) ) ) + {W}_{1 \times 1} * x ,$$

where

MHSA(X)=Concat(O₁, O₂, . . . , Oₕ)W^{o}, W_{3×3} represents a 3×3 convolution operation and a weight, and Concat represents a concatenation function.

Optionally, the fully connected layer is specified according to the following formula:

$${p}_{i} = \frac{{e}^{{z}_{i}}}{{\sum }_{j = 1}^{4} {e}^{{z}_{j}}} ,$$

where

pᵢ represents a probability of an i^{th} category, zᵢ represents an i^{th} element of a linear transformation output z, and e represents a natural constant.

According to a second aspect, this application provides an apparatus for determining endobronchial tuberculosis typing, including:
a dataset obtaining module, configured to obtain a dataset, where the dataset includes an endobronchial endoscopic image sample;
an endobronchial tuberculosis diagnostic model construction module, configured to construct an endobronchial tuberculosis diagnostic model, where the endobronchial tuberculosis diagnostic model is an endobronchial tuberculosis diagnostic model that is based on a ResNet34 framework and that incorporates multi-head self-attention and depthwise separable convolution;
a training module, configured to train the endobronchial tuberculosis diagnostic model based on the dataset; and
an endobronchial tuberculosis typing determining module, configured to input a bronchoscopy image of a user into a trained endobronchial tuberculosis diagnostic model to obtain the endobronchial tuberculosis typing.

According to a third aspect, this application provides a computer device, including a memory, a processor, and a computer program stored in the memory and executable on the processor, where the processor executes the computer program to perform steps of the method for determining endobronchial tuberculosis typing according to any one of the implementations.

According to specific embodiments provided in this application, this application discloses the following technical effects:

According to the method and apparatus for determining endobronchial tuberculosis typing, and the device provided in this application, depthwise separable convolution is introduced to split massive calculation in a traditional convolution operation into two small computational steps: depthwise convolution and pointwise convolution. Through the convolution operation in which a quantity of model parameters and an amount of computation are reduced, categorization performance of the model is improved, a training speed of the model is increased, and a computational burden of the model is effectively reduced. The second convolution in the second and third residual blocks in the residual module group 4 in the ResNet34 is replaced with the multi-head self-attention mechanism, so that the model can focus on both global and local features of the endobronchial image simultaneously, increasing the accuracy of the model to nearly 90%. In addition, dual universal serial bus (USB) foot pedals are used, so that an endobronchial tuberculosis artificial intelligence-assisted diagnostic system and a hospital bronchoscopy reporting system are ensured to be simultaneously used without interfering with each other. ResNet 34 is a convolutional neural network (CNN) architecture that is one of the ResNet family of models. ResNet 34 was proposed by Kaiming He et al. at Microsoft Research in 2015 and achieved excellent results in the ImageNet Large Scale Visual Recognition Competition (ILSVRC) that year. ResNet34 is characterized by its internal structure, which contains 34 convolutional layers, which are organized into Residual Blocks, each containing several convolutional layers and a shortcut connection. This design allows the network to mitigate the gradient vanishing problem while increasing depth, thus making it easier to optimize and improving accuracy. ResNet34 is an efficient and easy-to-train deep learning model with a wide range of applications in computer vision tasks such as image classification, target detection, and semantic segmentation.

### BRIEF DESCRIPTION OF THE DRAWINGS

To describe the technical solutions in the examples of this application or in the prior art more clearly, the following briefly describes the accompanying drawings required for the examples. Apparently, the accompanying drawings in the following description show merely some examples of this application, and a person of ordinary skill in the art may still derive other accompanying drawings from these accompanying drawings without creative efforts.

FIG. 1 is a schematic flowchart of a method for determining endobronchial tuberculosis typing according to an embodiment of this application;

FIG. 2 is a schematic diagram of an interface of an endobronchial tuberculosis artificial intelligence-assisted diagnostic system according to an embodiment of this application;

FIG. 3 is a schematic diagram of a working principle of an endobronchial tuberculosis artificial intelligence-assisted diagnostic system according to an embodiment of this application;

FIG. 4 is a schematic structural diagram of an endobronchial tuberculosis diagnostic model that is based on a ResNet34 framework and that incorporates multi-head self-attention and depthwise separable convolution;

FIG. 5 is a schematic structural diagram of a depthwise separable convolution residual block according to an embodiment of this application; and

FIG. 6 is a schematic structural diagram of a computer device according to an embodiment of this application.

### DETAILED DESCRIPTION OF THE EMBODIMENTS

The technical solutions in the embodiments of this application are clearly and completely described below with reference to the drawings in the embodiments of this application. Apparently, the described embodiments are only some rather than all of the embodiments of this application. All other embodiments obtained by a person of ordinary skill in the art based on the embodiments of the utility model without creative efforts shall fall within the protection scope of the utility model.

To make the above objectives, features, and advantages of the present disclosure more obvious and easier to understand, the present disclosure will be further described in detail with reference to the accompanying drawings and specific implementations.

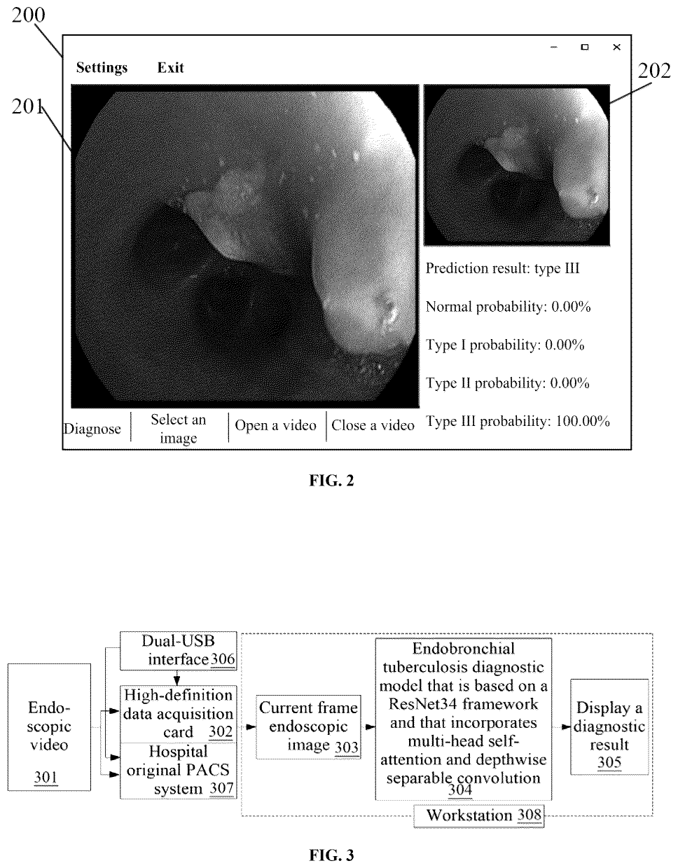

An endobronchial tuberculosis artificial intelligence-assisted diagnostic system is an intelligent diagnostic system 200 that is capable of identifying endobronchial tuberculosis and providing typing recommendations when used in cooperation with an endobronchial endoscopy, as shown in FIG. 2. A doctor only needs to open software and click an “open video” button, to synchronize a video detection area 201 in an upper left corner of the software with hospital's endoscopy workstation images. When a potential endobronchial tuberculosis lesion is found during endobronchial endoscopy on a patient, the doctor can acquire and send a current image to a hospital picture archiving and communication system (PACS) and the endobronchial tuberculosis artificial intelligence-assisted diagnostic system by stepping on a foot pedal. When an incoming signal from the medical foot pedal is detected by the endobronchial tuberculosis artificial intelligence-assisted diagnostic system, a current video frame is intercepted, an intercepted image 202 is displayed in an upper right corner of an endobronchial tuberculosis (EBTB) image diagnosis area, intelligent diagnosis is automatically performed on the image, and positive judgment and typing recommendations are displayed in a diagnostic result display area on a right side of a screen, as shown in FIG. 2. This helps the doctor in making diagnoses, and alleviates a burden of the doctor.

FIG. 3 is a schematic diagram of a working principle of an endobronchial tuberculosis artificial intelligence-assisted diagnostic system. An endoscopic video 301 is accessed into a high-definition data acquisition card 302 via a high-definition multimedia interface(HDMI) high-definition data cable. When a foot pedal is stepped on, a current frame image 303 of the video is captured and stored in a memory of a workstation 308. A trained endobronchial tuberculosis artificial intelligence-assisted diagnostic model 304 is configured to: read a current acquired image, automatically determine whether a diagnosis result is endobronchial tuberculosis, and automatically provide typing recommendations if the diagnosis result is endobronchial tuberculosis. If the diagnosis result is not endobronchial tuberculosis, Type 0 is indicated. In other words, a diagnostic result is displayed 305.

Dual universal serial bus (USB) foot pedals are adopted in the system, so that a high-definition data acquisition card of the endobronchial tuberculosis artificial intelligence-assisted diagnostic system, and original PACS image acquisition of the hospital are controlled through control signals of the foot pedals using a Dual-USB interface 306. The hospital PACS is a system utilized by the hospital for the doctors to acquire endobronchial data and provide diagnostic report. The hospital original PACS system 307 and the endobronchial tuberculosis artificial intelligence-assisted diagnostic system in this application are two independent systems without mutual interference.

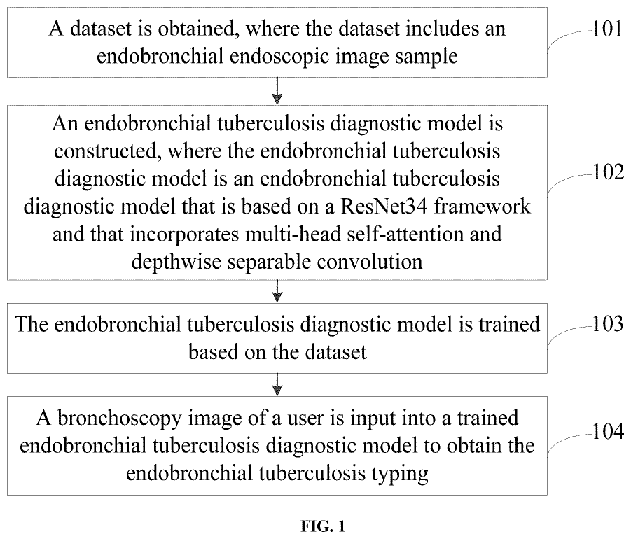

FIG. 1 is a schematic flowchart of a method for determining endobronchial tuberculosis typing according to an embodiment of this application. As shown in FIG. 1, the method includes the following steps.

In step 101, a dataset is obtained, where the dataset includes an endobronchial endoscopic image sample.

Specifically, clinical data is gathered from a hospital, and a database including over 20,000 endobronchial endoscopic image samples is constructed.

Then, preprocessing including scaling, normalization, and data enhancement is performed on the data, to balance a quantity of various endobronchial tuberculosis images in the database, and ensure a training effect.

In step 102, an endobronchial tuberculosis diagnostic model is constructed, where the endobronchial tuberculosis diagnostic model is an endobronchial tuberculosis diagnostic model that is based on a ResNet34 framework and that incorporates multi-head self-attention and depthwise separable convolution.

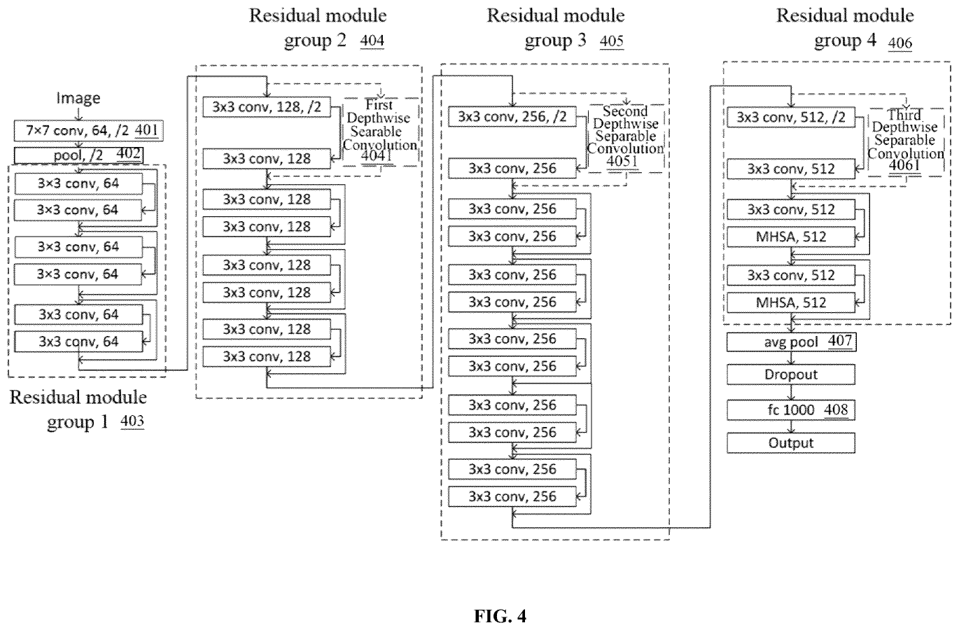

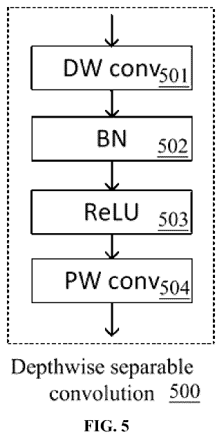

FIG. 4 is a schematic structural diagram of the endobronchial tuberculosis diagnostic model that is based on a ResNet34 framework and that incorporates multi-head self-attention and depthwise separable convolution. Specifically, the endobronchial tuberculosis diagnostic model includes:

A 7×7 convolutional layer 401, a pooling layer 402, a first residual module group 403, a second residual module group 404, a third residual module group 405, a fourth residual module group 406, a global average pooling layer 407, and a fully connected layer 408.

The 7×7 convolutional layer 401, the pooling layer 402, the first residual module group 403, the second residual module group 404, the third residual module group 405, the fourth residual module group 406, the global average pooling layer 407, and the fully connected layer 408 are sequentially connected.

The first residual module group 403 includes a first ordinary residual block, a second ordinary residual block, and a third ordinary residual block, where each of the first ordinary residual block, the second ordinary residual block, and the third ordinary residual block includes two 3×3 convolutional layers, and the first ordinary residual block, the second ordinary residual block, and the third ordinary residual block are sequentially connected.

The second residual module group 404 includes a first depthwise separable convolution residual block 4041, a fourth ordinary residual block, a fifth ordinary residual block, and a sixth ordinary residual block, where each of the fourth ordinary residual block, the fifth ordinary residual block, and the sixth ordinary residual block includes two 3×3 convolutional layers.

The first depthwise separable convolution residual block 4041, the fourth ordinary residual block, the fifth ordinary residual block, and the sixth ordinary residual block are sequentially connected.

The third residual module group 405 includes a second depthwise separable convolution residual block 4051, a seventh ordinary residual block, an eighth ordinary residual block, a ninth ordinary residual block, a tenth ordinary residual block, and an eleventh ordinary residual block, where each of the seventh ordinary residual block, the eighth ordinary residual block, the ninth ordinary residual block, the tenth ordinary residual block, and the eleventh ordinary residual block includes two 3×3 convolutional layers.

The second depthwise separable convolution residual block 4051, the seventh ordinary residual block, the eighth ordinary residual block, the ninth ordinary residual block, the tenth ordinary residual block, and the eleventh ordinary residual block are sequentially connected.

The fourth residual module group 406 includes a third depthwise separable convolution residual block 4061, a first multi-head self-attention mechanism residual block, and a second multi-head self-attention mechanism residual block.

The third depthwise separable convolution residual block 4061, the first multi-head self-attention mechanism residual block, and the second multi-head self-attention mechanism residual block are sequentially connected.

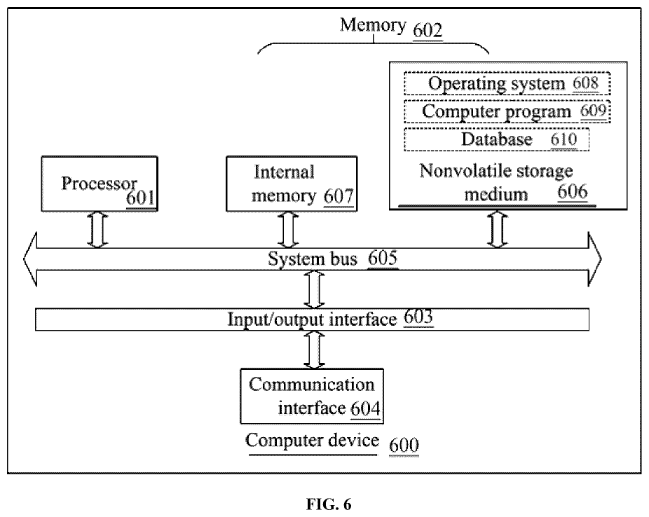

Refer to FIG. 5. Each depthwise separable convolution residual block 500 of the first depthwise separable convolution residual block, the second depthwise separable convolution residual block, and the third depthwise separable convolution residual block includes:
a DW conv layer 501, a BN layer 502, a ReLU layer 503, and a PW conv layer 504 that are sequentially connected.

The first ordinary residual block, the second ordinary residual block, the third ordinary residual block, the fourth ordinary residual block, the fifth ordinary residual block, the sixth ordinary residual block, the seventh ordinary residual block, the eighth ordinary residual block, the ninth ordinary residual block, the tenth ordinary residual block, and the eleventh ordinary residual block are all calculated according to the following formula:

$$y = F ( x , \{ {W}_{i} \} ) + {W}_{1 \times 1} * x , \qquad ( 1 )$$

where

y represents an output, F(x,{Wᵢ})=ReLU(BN(W₂*ReLU(BN(W₁*x)))), x represents an input, BN represents a batch normalization operation, ReLU represents a nonlinear activation operation, ReLU(x)=max(0,x). Max represents a maximum value taking operation, i=1 and 2 in Wᵢ, W₁ represents a first convolution operation and a weight, W₂ represents a second convolution operation and a weight, and W_{1×1} represents a 1×1 convolution operation and a weight.

The first depthwise separable convolution residual block, the second depthwise separable convolution residual block, and the third depthwise separable convolution residual block are all calculated according to the following formula:

$$y = F ( x , \{ {W}_{i} \} ) + {W}_{p w} * ( {W}_{d w} * x ) , \qquad ( 2 )$$

where

W_{pw} represents a pointwise convolution operation, and W_{dw} represents a depthwise convolution operation.

Both the first multi-head self-attention mechanism residual block, and the second multi-head self-attention mechanism residual block are calculated according to the following formula:

$$y = R e L U ( B N ( {W}_{3 \times 3} * M H S A ( X ) ) ) + {W}_{1 \times 1} * x , \qquad ( 3 )$$

where

MHSA(X)=Concat(O₁, O₂, . . . , Oₕ)W^{o}, W_{3×3} represents W_{1×1}, W_{1×1} represents a 3×3 convolution operation and a weight, Concat represents a concatenation function for parallel computing of a plurality of heads on different sub controls to obtain a merged self-attention output, MHSA(X)=Concat(O₁, O₂, . . . , Oₕ)W^{o}, and Oᵢ=Attention(O₁, O₁, O₁).

The fully connected layer is specified according to the following formula:

where

$${p}_{i} = \frac{{e}^{{z}_{i}}}{{\sum }_{j = 1}^{4} {e}^{{z}_{j}}} , \qquad ( 4 )$$

pᵢ represents a probability of an i^{th} category, zi represents an i^{th} element of a linear transformation output z, e represents a natural constant, z=Wh+b, h is an input feature vector with a dimension of 512, W is a weight matrix with a dimension of 4×512 of the fully-connected layer, and b is a bias vector with a dimension of 4 of the fully-connected layer, which is converted into a probability distribution by a Softmax function: p=Softmax(z).

In step 103, the endobronchial tuberculosis diagnostic model is trained based on the dataset.

A specific training process is as follows.

The endobronchial endoscopic image sample is input into the endobronchial tuberculosis diagnostic model to obtain a model output;
a difference between the model output and a true label is calculated by using a cross-entropy loss function to obtain a loss;
a gradient of the loss with respect to a parameter of the endobronchial tuberculosis diagnostic model is calculated, and the gradient of the loss is propagated from an output layer to an input layer through a chain rule; and
the parameter of the endobronchial tuberculosis diagnostic model is updated by using an optimizer based on the gradient of the loss, to obtain the trained endobronchial tuberculosis diagnostic model.

Model training parameters are configured as shown in Table 1:

TABLE 1
Configuration of model training parameters

| Parameters | Values |
|---|---|
| Input size | 224 × 224 |
| Epochs | 120 |
| Batch size | 32 |
| Optimizer | Adam |
| Loss function | Cross entropy loss |
| Learning rate | 1e-4 |
| Weight decay rate | 1e-4 |

In step 104, a bronchoscopy image of a user is input into a trained endobronchial tuberculosis diagnostic model to obtain the endobronchial tuberculosis typing.

Specifically, processing inside the endobronchial tuberculosis diagnostic model is as follows.

(1) A cony (7, 7 cin=3, cout=64, padding=3, stride=2) convolution operation is performed on 224×224 pixel image information through a 7×7 convolutional layer. Features of an image can be initially extracted in a larger field of view through the convolution operation, so that a size of the image is halved, and a depth of a feature map is increased to obtain a 64×112×112 feature matrix.

(2) The 64×112×112 feature matrix obtained in step (1) is input into a pooling layer for the conv (3,3 cin=3, cout=64, padding=1, stride=2) convolution operation, so that a size of the 64×112×112 feature matrix is halved to obtain a 64×56×56 feature matrix. The pooling layer is configured to: highlight obvious features and reduce an amount of computation of subsequent convolutional layers, improving overall computational efficiency of a network.

(3) The 64×56×56 feature matrix obtained in (2) is input into a residual module group 1. The module group includes three residual blocks, and each residual block has two 3×3 convolutional layers. An input and an output of each residual block are summed up to form a residual connection. A specific operation of each residual block is as shown in formula (1), and the 64×56×56 feature matrix is obtained by the module through operation. The module is mainly configured to: further extract and enhance features, and reduce a vanishing gradient problem through the residual connection.

(4) The feature matrix obtained in (3) is input into a residual module group 2 including four residual blocks, and each residual block includes two 3×3 convolutional layers, where a first residual block is a depthwise separable convolution residual block calculated according to formula (2), and the remaining three residual blocks are ordinary residual blocks are calculated according to formula (1). A 128×28×28 feature matrix is output through the residual module group 2. Compared with the feature extraction by the residual module group 1, the feature extraction at this stage is profound and intricate, so that more sophisticated features are learned through more residual blocks.

(5) The feature matrix obtained in (4) is input into a residual module group 3. The module includes three residual blocks, and each residual block includes two 3×3 convolutional layers, where a first residual block is a depthwise separable convolution residual block, a second residual block and a third residual block are calculated according to formula (2), and the remaining five residual blocks are ordinary residual blocks calculated according to formula (1). A 256×14×14 feature matrix is output through the residual module group 3. The residual module group 3 is a core part for feature extraction, and high-level features are extracted through a great number of residual blocks.

(6) The feature matrix obtained in (5) is input into a residual module group 4. The module includes three residual blocks, where a first residual block is a depthwise separable convolution residual block calculated according to formula (2). The remaining two residual blocks are residual blocks with multi-head self-attention (MHSA), that is, MHSA is introduced into second convolutional layers of the remaining two residual blocks, as calculated according to formula (3). The MHSA is introduced to capture a relationship and contextual information between distant features, enhancing a global performance of feature representation and improving feature expressiveness. A 512×7×7 feature matrix is output through the residual module group 4. The fourth residual module group is configured to: integrate and categorize the previously extracted features, thereby enabling the model to focus on both global and local features of the image, and improving the accuracy of the model.

(7) The feature matrix obtained in (6) is input into a global average pooling layer, and a 512×1×1 feature vector is output. The global average pooling layer is configured to: extract global features through dimensionality reduction, replace the fully connected layer, smooth a feature map, and enhance interpretability of the feature map.

(8) A categorizing task is achieved through the fully connected layer according to the feature vector output in (7), and typing is finally performed on an endobronchial tuberculosis image.

EBTB is categorized into six types according to the progression of the disease under tracheoscopy: inflammatory infiltration (type I), ulcerative necrosis (type II), granulation proliferation (type III), cicatricial stenosis (type IV), softening of tracheobronchial wall (type V), and lymph node fistula (type VI).

Based on a same inventive concept, an embodiment of this application further provides an apparatus for determining endobronchial tuberculosis typing for implementing the method for determining endobronchial tuberculosis typing. Implementation solutions provided by the apparatus for resolving the problems are similar with the implementation solutions recorded in the method, and therefore, specific limitations in apparatus embodiments for determining one or more pieces of endobronchial tuberculosis typing provided below, refer to the limitations on the foregoing method for determining endobronchial tuberculosis typing. Details are not described herein again.

In an exemplary embodiment, an apparatus for determining endobronchial tuberculosis typing is provided, including:
a dataset obtaining module, configured to obtain a dataset, where the dataset includes an endobronchial endoscopic image sample;
an endobronchial tuberculosis diagnostic model construction module, configured to construct an endobronchial tuberculosis diagnostic model, where the endobronchial tuberculosis diagnostic model is an endobronchial tuberculosis diagnostic model that is based on a ResNet34 framework and that incorporates multi-head self-attention and depthwise separable convolution;
a training module, configured to train the endobronchial tuberculosis diagnostic model based on the dataset; and
an endobronchial tuberculosis typing determining module, configured to input a bronchoscopy image of a user into a trained endobronchial tuberculosis diagnostic model to obtain the endobronchial tuberculosis typing.

In an embodiment, a computer device is provided. The computer device may be a server or a terminal, and an internal structure thereof may be as shown in FIG. 6. The computer device 600 includes a processor 601, a memory 602, an input/output (I/O) interface 603, and a communication interface 604. The processor 601, the memory 602, and the I/O interface 603 are connected through a system bus 605. The communication interface 604 is connected to the system bus 605 through the I/O interface 603. The processor 601 of the computer device 600 is configured to provide computing and control capabilities. The memory 602 of the computer device 600 includes a nonvolatile storage medium 606 and an internal memory 607. The nonvolatile storage medium 606 stores an operating system 608, a computer program 609, and a database 610. The internal memory 607 provides an environment for operation of the operating system 608 and the computer program 609 in the nonvolatile storage medium. The database 610 of the computer device 600 is configured to store data for determining endobronchial tuberculosis typing. The I/O interface 603 of the computer device 600 is configured to exchange information between the processor 601 and an external device. The communication interface 604 of the computer device 600 is configured to communicate with an external terminal through a network. The computer program 609 is executed by the processor 601 to implement a method for determining endobronchial tuberculosis typing.

Those skilled in the art may understand that the structure shown in FIG. 6 is only a block diagram of a part of the structure related to the solutions of this application and does not constitute a limitation on a computer device to which the solutions of this application are applied. Specifically, the computer device may include more or less components than those shown in the figure, or combine some components, or have different component arrangements.

In an example embodiment, a computer device is further provided, including a memory and a processor, where the memory stores a computer program, and the computer program is executed by the processor to implement the steps of the above method embodiment.

In an example embodiment, a computer-readable storage medium is provided. The computer-readable storage medium stores a computer program, and the computer program is executed by a processor to implement the steps of the above method embodiment.

In an example embodiment, a computer program product is provided. The computer program product includes a computer program, and the computer program is executed by a processor to implement the steps of the above method embodiment.

It is to be noted that information of a user (including but not limited to user device information, personal information of the user, and the like) and data (including but not limited to data for analysis, data for storage, data for exhibition and the like) in this application are information and data authorized by the user or fully authorized by each party, and relevant data shall be acquired, used, and processed according to relevant regulations.

Those of ordinary skill in the art may understand that all or some of the procedures in the method of the foregoing embodiments may be implemented by a computer program instructing related hardware. The computer program may be stored in a nonvolatile computer-readable storage medium. When the computer program is executed, the procedures in the embodiments of the foregoing method may be performed. Any reference to a memory, a storage, a database, or other media used in the embodiments of this application may include a nonvolatile and/or volatile memory. The nonvolatile memory may include a read-only memory (ROM), a magnetic tape, a floppy disk, a flash memory, an optical memory, a high-density embedded nonvolatile memory, a resistive random access memory (ReRAM), a magnetoresistive random access memory (MRAM), a ferroelectric random access memory (FRAM), a phase change memory (PCM), a graphene memory, etc. The volatile memory may include a random access memory (RAM) or an external cache memory. As an illustration rather than a limitation, the RAM may be in various forms, such as a static random access memory (SRAM) or a dynamic random access memory (DRAM).

The database in the embodiments of this application may include at least one of a relational database and a non-relational database. The non-relational database may include a distributed database based on a blockchain, but is not limited thereto. The processor in embodiments of this application may be a general processor, a central processor, a graphics processor, a digital signal processor, a programmable logic device, and a data processing logic device based on quantum computing, but is not limited thereto.

The technical characteristics of the above embodiments can be employed in arbitrary combinations. To provide a concise description of these embodiments, all possible combinations of all the technical characteristics of the above embodiments may not be described; however, these combinations of the technical characteristics should be construed as falling within the scope defined by the specification as long as no contradiction occurs.

Several examples are used herein for illustration of the principles and implementations of this application. The description of the foregoing examples is used to help illustrate the method of this application and the core principles thereof. In addition, those of ordinary skill in the art can make various modifications in terms of specific implementations and scope of application in accordance with the teachings of this application. In conclusion, the content of the present specification shall not be construed as a limitation to this application.

What is claimed is:

1. A method for determining endobronchial tuberculosis typing, comprising:
acquiring a dataset, by an endobronchial endoscopic, wherein the dataset comprises an endobronchial endoscopic image sample;
establishing an endobronchial tuberculosis diagnostic device comprising a memory and one or more processors through following steps, wherein the memory comprises an endobronchial tuberculosis diagnostic model:
constructing a primary endobronchial tuberculosis diagnostic model based on a ResNet34 framework and by incorporating multi-head self-attention and depthwise separable convolution; and
training the primary endobronchial tuberculosis diagnostic model based on the dataset to obtain the endobronchial tuberculosis diagnostic model; and
inputting, from the endobronchial endoscopic, a target bronchoscopy image of a target user into the endobronchial tuberculosis diagnostic device to output an endobronchial tuberculosis typing of the target user.

2. The method for determining endobronchial tuberculosis typing according to claim 1, wherein the training the endobronchial tuberculosis diagnostic model based on the dataset comprises the following steps:
inputting the endobronchial endoscopic image sample into the endobronchial tuberculosis diagnostic model to obtain a model output;
calculating a difference between the model output and a true label by using a cross-entropy loss function to obtain a loss;
calculating a gradient of the loss with respect to a parameter of the endobronchial tuberculosis diagnostic model, and propagating the gradient of the loss from an output layer to an input layer through a chain rule; and
updating, by an optimizer, the parameter of the endobronchial tuberculosis diagnostic model based on the gradient of the loss, to obtain the trained endobronchial tuberculosis diagnostic model.

3. The method for determining endobronchial tuberculosis typing according to claim 1, wherein the endobronchial tuberculosis diagnostic model comprises:
a 7×7 convolutional layer, a pooling layer, a first residual module group, a second residual module group, a third residual module group, a fourth residual module group, a global average pooling layer, and a fully connected layer, wherein
the 7×7 convolutional layer, the pooling layer, the first residual module group, the second residual module group, the third residual module group, the fourth residual module group, the global average pooling layer, and the fully connected layer are sequentially connected.

4. The method for determining endobronchial tuberculosis typing according to claim 3, wherein the first residual module group comprises a first ordinary residual block, a second ordinary residual block, and a third ordinary residual block, wherein each of the first ordinary residual block, the second ordinary residual block, and the third ordinary residual block comprises two 3×3 convolutional layers, and the first ordinary residual block, the second ordinary residual block, and the third ordinary residual block are sequentially connected;
the second residual module group comprises a first depthwise separable convolution residual block, a fourth ordinary residual block, a fifth ordinary residual block, and a sixth ordinary residual block, wherein each of the fourth ordinary residual block, the fifth ordinary residual block, and the sixth ordinary residual block comprises two 3×3 convolutional layers;
the first depthwise separable convolution residual block, the fourth ordinary residual block, the fifth ordinary residual block, and the sixth ordinary residual block are sequentially connected;
the third residual module group comprises a second depthwise separable convolution residual block, a seventh ordinary residual block, an eighth ordinary residual block, a ninth ordinary residual block, a tenth ordinary residual block, and an eleventh ordinary residual block, wherein each of the seventh ordinary residual block, the eighth ordinary residual block, the ninth ordinary residual block, the tenth ordinary residual block, and the eleventh ordinary residual block comprises two 3×3 convolutional layers;
the second depthwise separable convolution residual block, the seventh ordinary residual block, the eighth ordinary residual block, the ninth ordinary residual block, the tenth ordinary residual block, and the eleventh ordinary residual block are sequentially connected;
the fourth residual module group comprises a third depthwise separable convolution residual block, a first multi-head self-attention mechanism residual block, and a second multi-head self-attention mechanism residual block; and
the third depthwise separable convolution residual block, the first multi-head self-attention mechanism residual block, and the second multi-head self-attention mechanism residual block are sequentially connected.

5. The method for determining endobronchial tuberculosis typing according to claim 4, wherein the first ordinary residual block, the second ordinary residual block, the third ordinary residual block, the fourth ordinary residual block, the fifth ordinary residual block, the sixth ordinary residual block, the seventh ordinary residual block, the eighth ordinary residual block, the ninth ordinary residual block, the tenth ordinary residual block, and the eleventh ordinary residual block are all calculated according to the following formula:
$$y = F ( x , \{ {W}_{i} \} ) + {W}_{1 \times 1} * x ,$$
wherein
y represents an output, F(x,{Wᵢ})=ReLU(BN(W₂*ReLU(BN(W₁*x)))), x represents an input, BN represents a batch normalization operation, ReLU represents nonlinear calculation, in Wᵢ, i=1, 2, W₁ represents a first convolution operation and a weight, W₂ represents a second convolution operation and a weight, and W_{1×1} represents a 1×1 convolution operation and a weight.

6. The method for determining endobronchial tuberculosis typing according to claim 5, wherein the first depthwise separable convolution residual block, the second depthwise separable convolution residual block, and the third depthwise separable convolution residual block are all calculated according to the following formula:
$$y = F ( x , \{ {W}_{i} \} ) + {W}_{p w} * ( {W}_{d w} * x ) ,$$
wherein
W_{pw} represents a pointwise convolution operation, and W_{dw} represents a depthwise convolution operation.

7. The method for determining endobronchial tuberculosis typing according to claim 5, wherein both the first multi-head self-attention mechanism residual block, and the second multi-head self-attention mechanism residual block are calculated according to the following formula:
$$y = R e L U ( B N ( {W}_{3 \times 3} * M H S A ( X ) ) ) + {W}_{1 \times 1} * x ,$$
wherein
MHSA(X)=Concat(O₁, O₂, . . . , Oₕ)W^{o}, W_{3×3} represents a 3×3 convolution operation and a weight, and Concat represents a concatenation function.

8. The method for determining endobronchial tuberculosis typing according to claim 3, wherein the fully connected layer is specified according to the following formula:
$${p}_{i} = \frac{{e}^{{z}_{i}}}{{\sum }_{j = 1}^{4} {e}^{{z}_{j}}} ,$$
wherein
pᵢ represents a probability of an i^{th} category, zᵢ represents an i^{th} element of a linear transformation output z, and e represents a natural constant.

9. An apparatus for determining endobronchial tuberculosis typing, comprising:
an endobronchial endoscopic, configured to acquire an endobronchial endoscopic image sample to form a dataset; and
an endobronchial tuberculosis diagnostic device comprising one or more processors and a memory containing an endobronchial tuberculosis diagnostic model, wherein the endobronchial tuberculosis diagnostic model is established by following steps:
constructing a primary endobronchial tuberculosis diagnostic model based on a ResNet34 framework and by incorporating multi-head self-attention and depthwise separable convolution; and
training the primary endobronchial tuberculosis diagnostic model based on the dataset to obtain the endobronchial tuberculosis diagnostic model;
wherein a target bronchoscopy image of a target user is inputted into the endobronchial tuberculosis diagnostic device to output an endobronchial tuberculosis typing of the target user.

10. A computer device, comprising a memory, a processor, and a computer program stored in the memory and executable on the processor, wherein the processor executes the computer program comprising an endobronchial tuberculosis diagnostic model for:
receiving a dataset from an endobronchial endoscopic, wherein the dataset comprises an endobronchial endoscopic image sample;
constructing a primary endobronchial tuberculosis diagnostic model based on a ResNet34 framework and by incorporating multi-head self-attention and depthwise separable convolution;
training the primary endobronchial tuberculosis diagnostic model based on the dataset to obtain the endobronchial tuberculosis diagnostic model; and
receiving, from the endobronchial endoscopic, a target bronchoscopy image of a target user into the endobronchial tuberculosis diagnostic model to output an endobronchial tuberculosis typing of the target user.

11. The computer device according to claim 10, wherein the training the endobronchial tuberculosis diagnostic model based on the dataset comprises the following steps:
inputting the endobronchial endoscopic image sample into the endobronchial tuberculosis diagnostic model to obtain a model output;
calculating a difference between the model output and a true label by using a cross-entropy loss function to obtain a loss;
calculating a gradient of the loss with respect to a parameter of the endobronchial tuberculosis diagnostic model, and propagating the gradient of the loss from an output layer to an input layer through a chain rule; and
updating, by an optimizer, the parameter of the endobronchial tuberculosis diagnostic model based on the gradient of the loss, to obtain the trained endobronchial tuberculosis diagnostic model.

12. The computer device according to claim 10, wherein the endobronchial tuberculosis diagnostic model comprises:
a 7×7 convolutional layer, a pooling layer, a first residual module group, a second residual module group, a third residual module group, a fourth residual module group, a global average pooling layer, and a fully connected layer, wherein
the 7×7 convolutional layer, the pooling layer, the first residual module group, the second residual module group, the third residual module group, the fourth residual module group, the global average pooling layer, and the fully connected layer are sequentially connected.

13. The computer device according to claim 12, wherein the first residual module group comprises a first ordinary residual block, a second ordinary residual block, and a third ordinary residual block, wherein each of the first ordinary residual block, the second ordinary residual block, and the third ordinary residual block comprises two 3×3 convolutional layers, and the first ordinary residual block, the second ordinary residual block, and the third ordinary residual block are sequentially connected;
the second residual module group comprises a first depthwise separable convolution residual block, a fourth ordinary residual block, a fifth ordinary residual block, and a sixth ordinary residual block, wherein each of the fourth ordinary residual block, the fifth ordinary residual block, and the sixth ordinary residual block comprises two 3×3 convolutional layers;
the first depthwise separable convolution residual block, the fourth ordinary residual block, the fifth ordinary residual block, and the sixth ordinary residual block are sequentially connected;
the third residual module group comprises a second depthwise separable convolution residual block, a seventh ordinary residual block, an eighth ordinary residual block, a ninth ordinary residual block, a tenth ordinary residual block, and an eleventh ordinary residual block, wherein each of the seventh ordinary residual block, the eighth ordinary residual block, the ninth ordinary residual block, the tenth ordinary residual block, and the eleventh ordinary residual block comprises two 3×3 convolutional layers;
the second depthwise separable convolution residual block, the seventh ordinary residual block, the eighth ordinary residual block, the ninth ordinary residual block, the tenth ordinary residual block, and the eleventh ordinary residual block are sequentially connected;
the fourth residual module group comprises a third depthwise separable convolution residual block, a first multi-head self-attention mechanism residual block, and a second multi-head self-attention mechanism residual block; and
the third depthwise separable convolution residual block, the first multi-head self-attention mechanism residual block, and the second multi-head self-attention mechanism residual block are sequentially connected.

14. The computer device according to claim 13, wherein the first ordinary residual block, the second ordinary residual block, the third ordinary residual block, the fourth ordinary residual block, the fifth ordinary residual block, the sixth ordinary residual block, the seventh ordinary residual block, the eighth ordinary residual block, the ninth ordinary residual block, the tenth ordinary residual block, and the eleventh ordinary residual block are all calculated according to the following formula:
$$y = F ( x , \{ {W}_{i} \} ) + {W}_{1 \times 1} * x ,$$
wherein
y represents an output, F(x,{Wᵢ})=ReLU(BN(W₂*ReLU(BN(W₁*x)))), x represents an input, BN represents a batch normalization operation, ReLU represents nonlinear calculation, in Wᵢ, i=1, 2, W₁ represents a first convolution operation and a weight, W₂ represents a second convolution operation and a weight, and W_{1×1} represents a 1×1 convolution operation and a weight.

15. The computer device according to claim 14, wherein the first depthwise separable convolution residual block, the second depthwise separable convolution residual block, and the third depthwise separable convolution residual block are all calculated according to the following formula:
$$y = F ( x , \{ {W}_{i} \} ) + {W}_{p w} * ( {W}_{d w} * x ) ,$$
wherein
W_{pw} represents a pointwise convolution operation, and W_{dw} represents a depthwise convolution operation.

16. The computer device according to claim 14, wherein both the first multi-head self-attention mechanism residual block, and the second multi-head self-attention mechanism residual block are calculated according to the following formula:
$$y = R e L U ( B N ( {W}_{3 \times 3} * M H S A ( X ) ) ) + {W}_{1 \times 1} * x ,$$
wherein
MHSA(X)=Concat(O₁, O₂, . . . , Oₕ)W^{o}, W_{3×3} represents a 3×3 convolution operation and a weight, and Concat represents a concatenation function.

17. The computer device according to claim 12, wherein the fully connected layer is specified according to the following formula:
$${p}_{i} = \frac{{e}^{{z}_{i}}}{{\sum }_{j = 1}^{4} {e}^{{z}_{j}}} ,$$
wherein
pᵢ represents a probability of an i^{th} category, zᵢ represents an i^{th} element of a linear transformation output z, and e represents a natural constant.
